(19) United States
(12) Patent Application Publication
(10) Pub. No.: US 2006/0018975 A1
(43) Pub. Date: Jan. 26, 2006

(54) Methods and compositions for weight management and mood enhancement

(76) Inventor: Shawn M. Talbott, Draper, UT (US)

Correspondence Address: MACPHERSON KWOK CHEN & HEID LLP, 1762 TECHNOLOGY DRIVE, SUITE 226, SAN JOSE, CA, 95110, US

(21) Appl. No.: 10/895,253

(22) Filed: Jul. 20, 2004

(51) Int. Cl. A61K 36/752; A61K 36/82; A61K 33/26; A61K 31/56

(52) U.S. Cl. 424/646; 424/729; 424/736; 424/769; 514/171; 424/774

(57) ABSTRACT

The present invention provides nutritional supplement compositions and processes for treatment using nutritional supplements that assist in weight management. One aspect of the invention comprises the inclusion in a single supplement constituent(s) that assist in reduction of a subject's cortisol level, thermogenic constituent(s), and constituent(s) that assist in stabilizing blood sugar levels.

### CROSS-REFERENCE TO RELATED APPLICATIONS

Not applicable.

### BACKGROUND OF THE INVENTION

1. The Field of the Invention

The present invention is directed to treatments and nutritional supplements that assist in reduction of cortisol, cortisol effects, and assisting in weight loss and mood enhancement.

2. The Relevant Technology

There has been a large increase in the number of overweight and obese people in recent years. This leads to a number of illnesses, as well as psychological issues arising from people that are unhappy with their condition.

Many different treatments have been proposed, including exercise, diets, and nutritional supplements.

Some people have implicated cortisol in weight gain, and have proposed that ingestion of cortisol-reducing supplements can assist in controlling one's weight. Although there has been some very promising research regarding the benefits of reducing cortisol, much improvement is needed to find suitable nutritional supplements to assist with weight management.

### BRIEF SUMMARY OF THE INVENTION

The present invention provides nutritional supplement compositions and processes for treatment using nutritional supplements that assist in weight management.

One aspect of the invention comprises the inclusion in a single supplement constituent(s) that assist in reduction of a subject's cortisol level, thermogenic constituent(s), and constituent(s) that assist in stabilizing blood sugar levels.

Another aspect of the invention is a process for assisting in weight management comprising the ingestion of a suitable nutritional supplements with breakfast and with lunch.

These and other aspects of the present invention will become more fully apparent from the following description and appended claims, or may be learned by the practice of the invention as set forth hereinafter.

### BRIEF DESCRIPTION OF THE DRAWINGS

FIG. 1 is a graph showing a change in profile of mood states with respect to a group of subjects taking placebo and a nutritional supplement in accordance with one aspect of the present invention.

FIG. 2 is a graph showing a change in the salivary cortisol level with respect to a group of subjects taking placebo and a nutritional supplement in accordance with one aspect of the present invention.

FIG. 3A is a graph showing a change in body weight with respect to a group of subjects taking placebo and a nutritional supplement in accordance with one aspect of the present invention.

FIG. 3B is a graph showing a change in waist circumference with respect to a group of subjects taking placebo and a nutritional supplement in accordance with one aspect of the present invention.

FIG. 4A is a graph showing a change in fat mass and lean mass with respect to a group of subjects taking placebo and a nutritional supplement in accordance with one aspect of the present invention.

FIG. 4B is a graph showing a change in body fat with respect to a group of subjects taking placebo and a nutritional supplement in accordance with one aspect of the present invention.

### DETAILED DESCRIPTION OF THE PREFERRED EMBODIMENTS

It is an aspect of the present invention to provide improved nutritional supplements that are surprisingly effective for weight loss and in weight management. It is another feature of the present invention to provide improved nutritional supplements that are surprisingly effective in improving mood.

It is another aspect of the present invention to provide improved nutritional supplements that assist in reduction of cortisol levels. The hormone cortisol is important in small amounts to regulate carbohydrate metabolism, inflammation and cardiovascular function. It also plays a role in increasing blood pressure and blood sugar levels under some circumstances.

Unfortunately, there is now evidence that increased levels of cortisol leads to an increase in abdominal fat accumulation. An increased cortisol level has also been implicated in a disruption of energy metabolism that can result in the development of a condition known as Metabolic Syndrome X. In studies of stress and cortisol exposure, high cortisol secretion has been associated specifically with abdominal fat, and abdominal fat has in turn been associated with illness and death from cardiovascular disease, diabetes, hypertension, hyperlipidemia, hyperinsulinemia, and insulin resistance. Women with a high cortisol response, compared to low responders, in evaluations of stress response, have been shown to consume more total calories, more calories from sweet and fatty foods (“comfort” foods), and report more negative moods. Hence, although cortisol is an important hormone having a very important function in healthy adults, abnormally high levels of cortisol are unhealthy.

Even the stress of dieting and dietary restraint has been associated with elevations in cortisol. This makes traditional dieting or dietary restraint counterproductive and difficult to maintain.

In accordance with the present invention, a particularly useful composition suitable for reduction of cortisol levels has been identified. In one embodiment, this composition includes an extract of magnolia bark, L-theanine, and beta-sitosterol. A currently preferred composition contains 100 mg of magnolia bark extract that is standardized to contain 1.5% honokiol; 25 mg of L-theanine (98% pure); and 30 mg of beta-sitosterol. It is preferred that this composition be taken twice a day, once with breakfast and again with lunch.

It will be understood by those of ordinary skill in the art that other substances that result in a reduction of cortisol may be used in place of those listed above. For example, other suitable cortisol modulating ingredients include, without limitation, Ashwagandha, Epimedium, Garlic, and Phosphatidylserine. Various cortisol modulating substances may be used individually or in combination with other substances.

Moreover, the source and amount of each cortisol modulating substance may be selected in order to provide a particular product. For example, each cortisol modulator may be obtained from natural sources and presented in a low to moderate dosage, in order to provide a natural product that can be used as a holistic treatment or nutritional supplement. Cortisol modulators from either natural or synthetic sources may be used in higher concentrations to impart a potency that is suitable for use in as a prescription medicine. Those of ordinary skill in the art will be able to determine a suitable therapeutically effective amount on an individual by individual basis. However, in one aspect of the present invention, the cortisol modulator component of the formulation may be included in an amount of from about 2% to about 100% of the formulation. In another aspect, the amount may be from about 10% to about 50% of the formulation.

It is another aspect of the invention to combine stress-reducing substances with the cortisol modulating substance(s). A number of other bioactive agents have been discovered to impart, or contribute to, a general stress reducing effect. For example, many agents impart an anti-anxiety (anxiolytic), or sedative effect. Such anti-stress ingredients include without limitation, certain branched chain amino acids, such as leucine, isoleucine, and valine, tyrosine, ginseng, glutamine, suma (i.e. Brazilian ginseng), schisandra, rhodiola, astragalus (i.e. Indian ginseng), vitamin C, magnesium, B-complex vitamins, such as thiamin, riboflavin, pyridoxine, and pantothenic acid, kava, melatonin, valerian, and gotu kola. Such anti-stress agents may optionally be included in the formulation of the present invention with the cortisol modulator ingredient. The specific type and amount of anti-stress ingredient, or mixture of anti-stress ingredient will depend on the specific properties desired for the final formulation. Those of ordinary skill in the art will be able to determine therapeutically effective amounts for specific ingredients based on the other ingredients in the formulation, and the mode of administration. Various combinations of such ingredients may be made, or individual ingredients may be selected and included in the present formulation, in order to achieve a specifically desired result. However, in one aspect, the amount of anti-stress ingredient used in the formulation may be from about 1% to about 50% of the formulation. In another aspect, the amount of anti-stress ingredient may be from about 5% to about 30% of the formulation.

Various positive health benefit imparting agents may optionally be included with the cortisol modulator in the formulation of the present invention. Examples of such agents include without limitation, water and oil soluble vitamins, various amino acids, minerals, including ionic minerals, enzymes, and other herbal extracts containing a number of active ingredients, such as antioxidants. Water soluble vitamins that may be included in the formulation of the present invention include, without limitation, vitamin B1, B2, B3, B5, B6, B12, B13, B15, B17, biotin, choline, folic acid, inositol, para-aminobenzoic acid (PABA), vitamin C, and vitamin P. Additionally, oil soluble vitamins that may be used include without limitation, vitamin A, vitamin D, vitamin E, and vitamin K. Amino acids other than those recited above, may include without limitation, alanine, arginine, carnitine, gamma-aminobutyric acid (GABA), glutamine, glycine, histidine, lysine, methionine, N-acetyl cysteine, ornithine, phenylalanine, and taurine. Ionic minerals that may be use include both anionic and cationic minerals. Further, examples of suitable anti-oxidants include without limitation, tea extract, such as green tea, oolong, and black teas, grape seed, beta-carotene, and co-enzyme Q-10. Specific examples of beneficial enzymes include bromelain and papain, as well as others. The specific type and amount of positive health benefit imparting agent will be selected depending on the qualities or properties desired of the final formulation. However, in one aspect, the amount of positive health benefit imparting agent included may be from about 0.1% w/w to about 30% w/w of the formulation. In another aspect, the amount of positive health benefit imparting agent may be from about 5% to about 10% of the formulation.

It is another aspect of the present invention to provide a useful composition for obtaining thermogenic action in combination with cortisol modulation substance(s) in order to provide a composition that is capable of elevating basal metabolic rate (BMR) without also elevating cortisol levels. Such a composition provides increased efficacy and safety, as compared to the administration of thermogenic ingredients alone. In one embodiment, a thermogenic composition includes an extract of green tea leaf extract and bitter orange peel. A currently preferred composition contains 100 mg of green tea extract that has been standardized to contain 50% epigallochatechingallate (EGCG) and 25 mg of bitter orange extract that has been standardized to contain 5% synephrine. It is preferred that this composition be taken twice a day, once with breakfast and again with lunch.

Other thermogenic substances may be used in place of or in combination with those identified above. However, many thermogenic agents, such as ephedra, including ephedrine and pseudoephedrine, caffeine, yohimbe, and coleus forskohli, substantially elevates various physiologic stress indicators, such as serum cortisol levels. Such stress indicators generally have a negative effect on the results sought through administration of such thermogenic agents, such as increased weight loss, or weight management. For example, sustained elevated cortisol levels facilitates weight gain, and accumulation of fat in the face, chest, and abdominal region as noted above. This action, therefore, reduces the effectiveness of the thermogenic agent. As another example, excessive cortisol levels increase the catabolism of lean muscle tissue. As lean muscle tissue burns calories and adds to the overall metabolism of the body, reducing muscle with elevated cortisol levels defeats the objective of thermogenic compounds to a degree, by reducing the overall metabolism rate achieved. The inclusion of cortisol modulating substances has been discovered to allow the use of various thermogenic substances without these side effects.

In another aspect of the present invention, one or more cortisol modulators may be combined with one or more ingredients for control of blood sugar, in order to provide a composition that is capable of elevating basal metabolic rate, controlling appetite, and increasing fat oxidation, without also elevating cortisol levels. Such a composition provides increased efficacy and safety, as compared to the administration of blood sugar controlling ingredients alone. In one embodiment, a blood sugar controlling composition contains an extract of banaba leaf and vanadium. A currently preferred composition contains 16 mg of banaba leaf extract standardized to contain 1% of corosolic acid, and 5 mcg of vanadium. It is preferred that this composition be taken twice a day, once with breakfast and again with lunch.

Other agents for enhancing control of blood sugar metabolism include alpha lipoic acid, cinnamon, chromium, and ginseng.

It has been found to be surprisingly effective to co-administer a cortisol modulator with a thermogenic agent and/or with an agent for controlling blood sugar. Such a combination has been found to effectively enhance the action of the thermogenic and blood sugar controlling agents, and to reduce or eliminate many of the adverse side effects thereof. In addition, other anti-stress ingredient or positive health benefit imparting ingredients may be added in order to achieve a specifically desired result. The cortisol modulator should be presented in a therapeutically effective amount, and may be selected from any of the cortisol modulators as disclosed herein. However, in one aspect, the cortisol modulator may be theanine. In another aspect, the cortisol modulator may be magnolia bark. In yet another aspect, the cortisol modulator may be beta-sitosterol.

The compositions of the present invention may be formulated for administration through various known routes of administration, such as oral, parenteral, and transdermal routes. Examples of oral dosage formulations include without limitation, tablets, capsules, liquids, suspensions, powders, effervescent beverages, lozenges, chewing gum, candy, etc. Examples of transdermal routes of administration include without limitation, topical formulations, such as lotions, creams, gels, and pastes, and transdermal patches, such as liquid reservoir patches, plasters, and adhesive matrix patches. Suitable ingredients required to produce a particular formulation, such as specific carriers, excipients, binders, penetration enhancers, etc., will be readily recognized by those of ordinary skill in the art.

The foregoing features and advantages of the invention will become apparent be reference to the following examples:

### EXAMPLE 1

A combination of cortisol modulating substances with thermogenic substances and blood-sugar level controlling substances was found to lead to unexpectedly positive results for weight loss and weight and body management, and for providing a greater sense of well-being in an experiment involving 50 volunteers recruited to participate in a 12-week weight loss program (42 women and 8 men). These volunteers were randomly assigned to receive the supplement (S, n=25) or a matching placebo (P, n=25) in a double-blind fashion. Recruits were selected based upon self-reported high to moderate levels of stress and frustration/dissatisfaction with previous weight loss attempts. This resulted in a test group of people who had tried and failed to lose weight on other programs. All participants were currently participating in a regular exercise program (at least 3 days weekly) and had attempted one or more weight loss diets within the past year. Forty-two subjects completed the 12-week study (P, n=17, 15 women and 2 men; S, n=25, 19 women and 6 men). Information regarding the members of this study is shown in Table 1.

TABLE 1
Subject characteristics (N = 50)

| Group | Age (y) | BW (kg) | BF (%) | Waist (cm) |
|---|---|---|---|---|
| Placebo (P, n = 25) | 44 ± 7 | 75.9 ± 13.6 | 28.4 ± 6.3 | 84 ± 13 |
| Supplement | 46 ± 9 | 76.8 ± 13.2 | 29.1 ± 5.3 | 89 ± 15 |
| (S, n = 25) | | | | |

Abbreviations:
BW = Body weight;
BF = Body fat;
Waist = Waist circumference

Supplement or placebo was provided to each subject in a randomized and blinded fashion on a monthly basis. The supplement used in this study had the formulation of Table 2 on a per capsule basis:

TABLE 2

| | |
|---|---|
| Magnolia bark extract (1.5% honokiol) | 100 mg |
| L-Theanine (98%) | 25 mg |
| Beta-sitosterol (50%) | 30 mg |
| Green tea extract (50% EGCG) | 100 mg |
| Bitter orange extract (5% synephrine) | 25 mg |
| Banaba leaf extract (1% corosolic acid) | 16 mg |
| Vanadium | 5 mcg |
| Calcium | 150 mg |
| Vitamin C | 100 mg |

Subjects consumed 2 capsules per day of the supplement of Table 2 and look-alike placebo capsules, one at breakfast and the second at lunch. The test ran for 12 weeks while the subjects also adhered to a moderate diet and exercise program.

Diets were based on resting metabolic rate (RMR, MetaCheck, Korr Medical Technologies, Salt Lake City, Utah) and exercise regimens were customized for each participant by a certified personal trainer (5 d/wk, 3 d aerobic+2 d strength) to result in no more than 500 kcal/d deficit. Subjects met as a group on a bimonthly basis for educational lectures on diet, exercise, and metabolism.

Before and after the 12-week weight loss program, measurements were made of body weight (TBF-300A scale, Tanita, Arlington Heights. IL), body fat by bioelectrical impedance analysis (BIA, Quantum II, RJL Medical Systems, Clinton Township, Mich.), waist circumference, profile of mood states (POMS), and salivary cortisol (Salimetrics, State College, Pa.). Saliva was collected using cotton salivettes within 15 minutes of morning waking and frozen at −20 degrees C. until analysis in duplicate.

Paired Student's t-tests were used to assess differences between pre-post measures. Students t-tests were used to compare the relative (percentage) change from pre-post between the two groups (S and P). Statistical significance was accepted at P<0.05. Data are expressed as mean+SD.

Results of this study are shown in Table 3.

TABLE 3
Key Outcomes

| Group | BW (kg) | BF (%) | Waist (cm) | Mood | Cortisol (ug/dL) |
|---|---|---|---|---|---|
| P, pre | 75.9 ± 13.6 | 28.4 ± 6.3 | 83.8 ± 12.7 | 139 ± 22 | 0.494 ± 0.212 |
| P, post | 75.4 ± 13.2 | 26.4 ± 5.6 | 81.3 ± 12.7 | 127 ± 48 | 0.473 ± 0.163 |
| % change | −0.7% | −7.0%* | −2.9% | +8.6%* | −4.2% |
| S, pre | 76.8 ± 13.2 | 29.1 ± 5.3 | 88.9 ± 15.2 | 134 ± 19 | 0.457 ± 0.228 |
| S, post | 72.7 ± 12.7 | 24.0 ± 3.9 | 81.3 ± 12.7 | 114 ± 26 | 0.363 ± 0.159 |
| % change | −5.3%*# | −17.5%*# | −8.5%* | −14.9%*# | −20.6%*# |

Abbreviations:
BW = Body weight;
BF = Body fat;
Waist = Waist circumference;
Mood = Global Mood States (POMS);
Cortisol = Waking salivary cortisol
P = Placebo,
S = Supplement
*= P < 0.05 compared to Pre
#= P < 0.05 compared to P

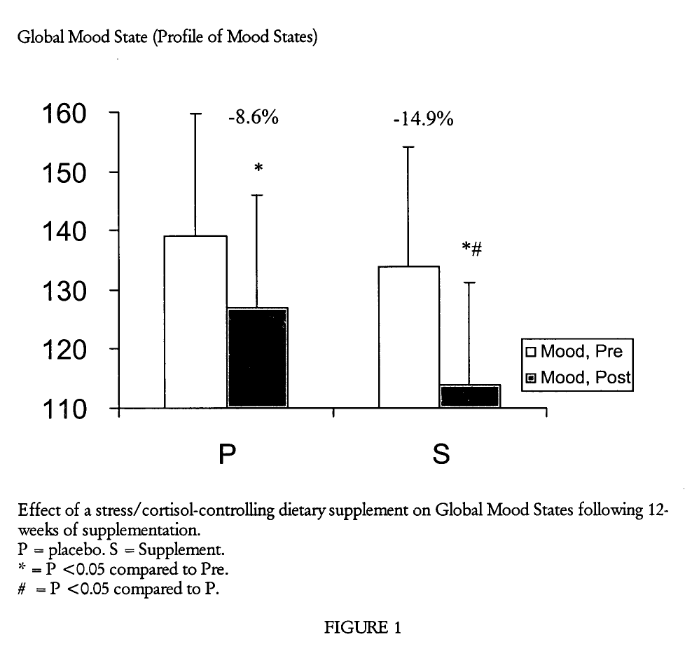

FIG. 1 is a graph showing a change in profile of mood states with respect to the group of subjects taking placebo and the supplement. This study showed that compared to P (placebo, diet and exercise), the Supplement-control group (S) had a greater improvement in global mood state (an index of tension, depression, fatigue and overall stress) of +14.9% versus only +8.6% for P.

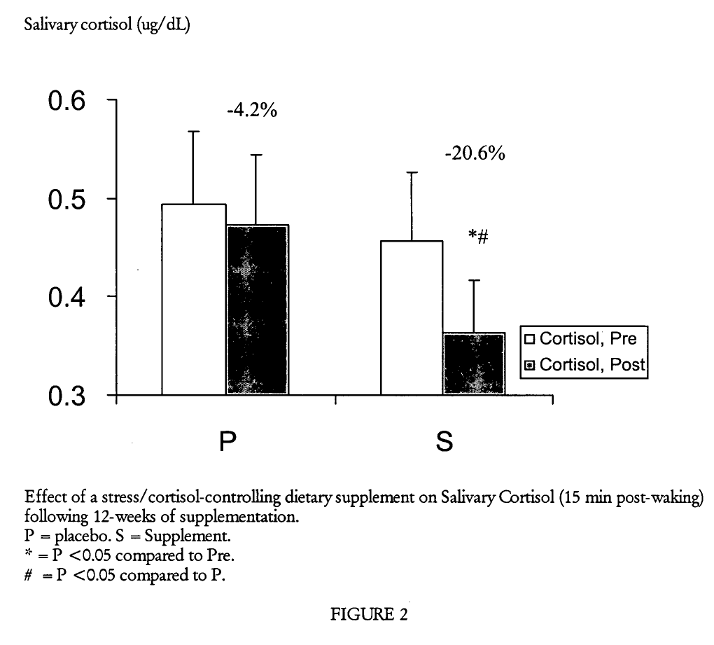

FIG. 2 is a graph showing a change in the salivary cortisol level with respect to the test group. Subjects taking the supplement demonstrated significantly reduced cortisol exposure (−20.6% versus −4.2%) with respect to those taking the placebo.

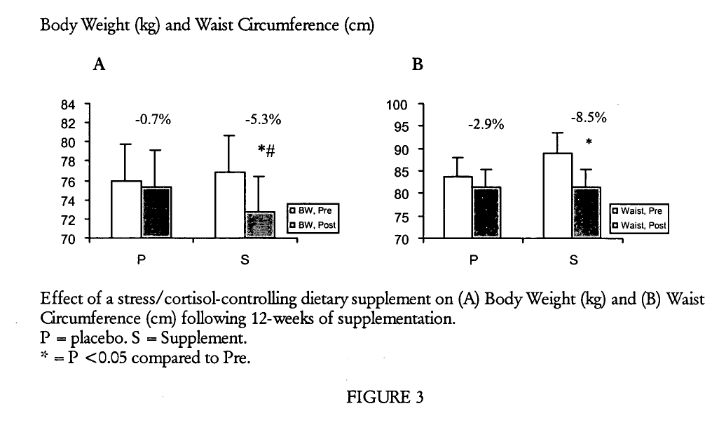

FIG. 3A is a graph showing a change in body weight with respect to the test subjects. Those taking the supplement showed a greater loss of body weight (−5.3% versus −0.7%).

FIG. 3B is a graph showing a change in waist circumference among the test subjects. Those taking the supplement exhibited a greater reduction in waist circumference than those taking the placebo (−8.5% versus −2.9%).

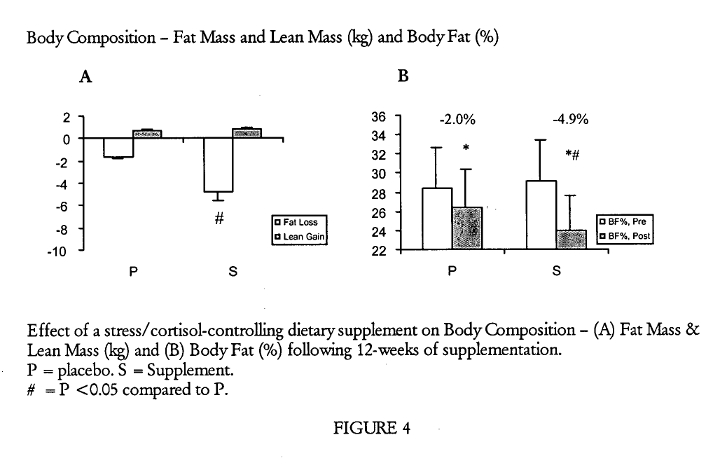

FIGS. 4A and 4B show the greater reduction in total and percent body fat among the supplement-control group versus the placebo group (−4.9% versus −2.0%).

Although it was expected that the supplement would have benefits over a placebo, the actual results went far beyond those that had been contemplated.

It is interesting to note that the difference in dropout rate between groups (32% from P and none from S) suggests that the “anti-stress” effects of the supplement may have made the diet/exercise regimen more tolerable. It is believed that a normal dropout rate from weight loss programs lasting 8 weeks or more is about 20 to 30%.

Although the magnitude of weight loss was less than 0.5-kg per week in the cortisol-control (supplement) group, this amount (4.1 kg over 12 weeks) is a meaningful percent loss (−5.3%) in this group of moderately fit, regular exercisers with a history of previous weight loss attempts. The fact that these “tough-cases” could be helped with weight loss by adding the supplement of Table 2 to the diet suggests that other populations who have struggled with their weight or who have tried/failed on other popular weight loss programs, may be able to benefit from a similar regimen.

Although the present supplement of Table 2 was listed to contain specific amounts of specific substances, it will be appreciated by those of ordinary skill in the art after reviewing the information and teachings contained herein that changes may be made in the actual constituents disclosed herein and in the amounts of the specific constituents of the compositions disclosed herein with an expectation that the desirable effects of that supplement may be maintained. For example, it would be expected that one could vary the amount of the various substances upwardly or downwardly and still obtain the beneficial effects disclosed herein to a greater or lesser degree. It would also be expected by one of ordinary skill in the art in view of the teachings herein that one could substitute other constituents having cortisol-lowering, and/or thermogenic; and or blood sugar controlling properties in place of the specific materials listed in Table 2, and still obtain meaningful beneficial effects.

### EXAMPLE 2

The following cortisol modulating composition has been determined to be effective in controlling the cortisol level of subjects:

| Formulation | Amount in % w/w |
|---|---|
| Magnolia bark extract (Magnolia | 65% |
| Officinalis) | |
| Beta-Sitosterol | 19% |
| L-Theanine | 16% |

### EXAMPLE 3

The following combination of cortisol modulating, thermogenic and blood sugar controlling substances has been determined to be effective in effecting weight loss and mood elevation among subjects:

| Formulation | Amount in % w/w |
|---|---|
| Calcium | 20% |
| Green tea leaf (Camellia sinensis) | 19% |
| Vitamin C | 19% |
| Beta-Sitosterol | 10% |
| Magnolia bark extract (Magnolia | 10% |
| Officinalis) | |
| L-Theanine | 10% |
| Panax ginseng root | 5% |
| Banaba leaf (Lagerstroemia speciosa) | 5% |
| Chromium | 1% |
| Vanadium | 1% |

### EXAMPLE 4

The following formulation is prepared and administered to subjects, containing cortisol modulating, thermogenic, and blood sugar controlling agents, and will result in weight reduction, weight control, and/or mood elevation:

| Formulation | Amount in % w/w |
|---|---|
| Magnolia bark extract (Magnolia | 80% |
| Officinalis) | |
| Green tea leaf (Camellia sinensis) | 10% |
| Alpha lipoic acid | 10% |

### EXAMPLE 5

The following formulation is prepared and administered to subjects, containing cortisol modulating, thermogenic, and blood sugar controlling agents, and will result in weight reduction, weight control, and/or mood elevation:

| Formulation | Amount in % w/w |
|---|---|
| Banaba leaf (Lagerstroemia speciosa) | 25% |
| Asian ginseng (Panax ginseng) | 25% |
| Green tea leaf (Camellia sinensis) | 25% |
| L-Theanine | 25% |

### EXAMPLE 6

The following formulation is prepared and administered to subjects, containing cortisol modulating agents, and will result in reduction of cortisol levels:

| Formulation | Amount in % w/w |
|---|---|
| Magnolia bark extract | 75% |
| (Magnolia Officinalis) | |
| L-Theanine | 25% |

### EXAMPLE 7

The following formulation is prepared and administered to subjects, containing cortisol modulating and thermogenic agents, and will result in weight loss and/or weight control:

| Formulation | Amount in % w/w |
|---|---|
| Bitter orange (Citrus aurantium) | 60% |
| Green tea extract (Camellia sinensis) | 20% |
| L-Theanine | 20% |

### EXAMPLE 8

The following formulation is prepared and administered to subjects, containing cortisol modulating, thermogenic, and blood sugar controlling agents, and will result in weight reduction, weight control, and/or mood elevation:

| Formulation | Amount in % w/w |
|---|---|
| Guarana (Paullinia cupana) | 60% |
| Beta-Sitosterol | 30% |
| Alpha lipoic acid | 10% |

### EXAMPLE 9

The following formulation is prepared and administered to subjects, containing cortisol modulating and thermogenic agents, and will result in weight loss and/or weight control:

| Formulation | Amount in % w/w |
|---|---|
| Bitter Orange (Citrus aurantium) | 50% |
| Beta-Sitosterol | 30% |
| Phosphatidylserine | 20% |

### EXAMPLE 10

The following formulation is prepared and administered to subjects, containing cortisol modulating, thermogenic, and blood sugar controlling agents, and will result in weight reduction, weight control, and/or mood elevation:

| Formulation | Amount in % w/w |
|---|---|
| Banaba leaf extract (Lagerstroemia | 34% |
| speciosa) | |
| Magnolia bark extract (Magnolia | 33% |
| Officinalis) | |
| Green tea extract (Camellia sinensis) | 33% |

### EXAMPLE 11

The following formulation is prepared and administered to subjects, containing cortisol modulating and thermogenic agents, and will result in weight loss and/or weight control:

| Formulation | Amount in % w/w |
|---|---|
| Green tea extract (Camellia sinensis) | 40% |
| Ashwagandha (Withania somnifera) | 20% |
| Kava kava (Piper methysticum) | 20% |
| Valerian (Valeriana officinalis) | 20% |

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A nutritional supplement for weight management, comprising:
a. at least one substance that will result in a lowering of cortisol;
b. at least one substance that will result in a thermogenic function; and
c. at least one substance that will assist in controlling the blood sugar level.

2. A nutritional supplement for use in weight management, comprising:
a. magnolia bark extract;
b. L-theanine;
c. Beta-sitosterol;
d. green tea extract;
e. bitter orange extract;
f. banaba leaf extract; and
g. vanadium.

3. A nutritional supplement for use in weight management, comprising:
a. 100 mg of magnolia bark extract containing 1.5% honokiol;
b. 25 mg of L-theanine (98%);
c. 30 mg of Beta-sitosterol (50%);
d. 100 mg of green tea extract containing 50% epigallochatechingallate;
e. 25 mg of bitter orange extract containing 5% synephrine;
f. 16 mg of banaba leaf extract containing 1% corosolic acid;
g. 5 mcg of vanadium;
h. 150 mg of calcium; and
i. 100 mg of vitamin C.

4. A process for assisting in weight management, comprising the steps of:
a. obtaining a nutritional supplement comprising:
1. 100 mg of magnolia bark extract containing 1.5% honokiol;
2. 25 mg of L-theanine (98%);
3. 30 mg of Beta-sitosterol (50%);
4. 100 mg of green tea extract containing 50% epigallochatechingallate;
5. 25 mg of bitter orange extract containing 5% synephrine;
6. 16 mg of banaba leaf extract containing 1% corosolic acid;
7. 5 mcg of vanadium;
8. 150 mg of calcium; and
9. 100 mg of vitamin C;
b. consuming one dose of said supplement with breakfast; and
c. consuming one dose of said supplement with lunch.

5. A cortisol reducing formulation, comprising:
a. a therapeutically effective amount of magnolia bark extract;
b. a therapeutically effective amount of L-theanine; and
c. a therapeutically effective amount of Beta-sitosterol.

6. A cortisol reducing formulation, comprising:
a. 100 mg of magnolia bark containing 1.5% honokiol;
b. 25 mg of L-theanine (98%); and
c. 30 mg beta-sitosterol (50%).
